(19) United States
(12) Patent Application Publication
(10) Pub. No.: US 2005/0201985 A1
(43) Pub. Date: Sep. 15, 2005

(54) Modified adenoviral vectors for use in vaccines and gene therapy

(76) Inventors: Stefan Kostense, Den Haag (NL); Olga Johanna Alberdina Elisa Ophorst, Alphen aan den Rijn (NL); Menzo Jans Emco Havenga, Alphen aan den Rijn (NL)

Correspondence Address: TRASK BRITT, P.O. BOX 2550, SALT LAKE CITY, UT, 84110, US

(21) Appl. No.: 11/064,910

(22) Filed: Feb. 24, 2005

Related U.S. Application Data
(63) Continuation of application No. PCT/NL02/00608, filed on Sep. 20, 2002.

(51) Int. Cl. A61K 48/00; C12N 5/08; C12N 15/86

(52) U.S. Cl. 424/93.2; 435/456; 435/372

(57) ABSTRACT

The present invention provides novel methods and means for influencing the CTL-sensitivity of antigen presenting cells (such as dendritic cells) upon viral infections. The invention provides gene delivery vehicles useful in different therapeutic settings such as vaccination and/or gene therapy.

### CROSS-REFERENCE TO RELATED APPLICATION

This application is a continuation of PCT International Patent Application No. PCT/NL2002/000608, filed on Sep. 20, 2002, designating the United States of America, and published, in English, as PCT International Publication No. WO 2004/027073 A1 on Apr. 1, 2004, the contents of the entirety of which is incorporated by this reference.

### TECHNICAL FIELD

The invention relates to the fields of biotechnology and medicine and, in particular, to the field of vaccination using modified adenoviral vectors. The invention further relates to the field of gene therapy and to methods and means for altered immune responses in subjects treated with viral vectors, such as adenoviruses, carrying a transgene.

### BACKGROUND

Examples of currently available vaccines include life-attenuated viruses, whole inactivated viruses, subunit vaccines (either or not based on recombinant protein), peptide vaccines, (naked) DNA-vaccines, and immunogen-carriers such as Modified Vaccinia viruses (MVA), recombinant adenoviruses, poxviruses or alphaviruses. Since for many human diseases it is unknown which component of the immune systems affords protection (antibodies and/or T-cells), the ideal vaccine should trigger both potent cellular and humoral responses. Adenovirus is favored by many in the art because it can be produced to high titers, it has consistently proven to be a highly efficient vaccine vehicle against various pathogens (Bruna-Romero et al. 2001; Sullivan et al. 2000), and in direct comparative studies with for instance MVA, adenovirus was identified as being superior for the induction of immunity and protection upon challenge in a non-human primate HIV model (Shiver et al. 2002). Moreover, adenovirus seems to induce both humoral (antibodies) and cellular (cytotoxic T-lymphocytes) immune responses against inserted antigens or proteins encoded by encompassed nucleic acid (Bruna-Romero et al. 2001; Shi et al. 2001; Shiver et al. 2002; Sullivan et al. 2000). Thus, insertion of genes encoding antigenic proteins derived from different kinds of pathogenic entities (viruses, bacteria, etc.) or through the use of tumor-specific proteins (tumor-vaccination) into recombinant adenovirus should in principle prime the host immune system towards these antigens resulting in protection upon infection of the pathogenic entity (prophylactic vaccination) or eradication of diseased and/or infected cells (therapeutic vaccination).

Besides their potential role in vaccines, adenoviruses are extensively used in gene therapy settings. Examples of other viral vector systems used in both therapeutic applications are alphaviruses (such as Semliki Forest virus, Sindbis virus and Venezuelan Equine Encephalitis virus), Human Immunodeficiency virus (HIV), poxvirus and influenza viruses. One of the problems that are encountered when adenoviruses are used is related to the fact that a large part of the human population during life has encountered several adenovirus infections, resulting in (high) titers of neutralizing antibodies against many of the different serotypes. One of the major tasks recognized in the art was to circumvent this problem and to provide (recombinant) adenoviruses that would be neutralized significantly less than the generally used adenoviruses (such as Ad2, Ad3, Ad5, Ad7 and Ad12) and that still would infect the target cell of interest. Although many solutions to these problems have been provided (for instance by using chimeric adenoviruses or poorly-neutralized serotypes (PCT International Publications WO 00/03029, WO 02/24730, and WO 00/70071, the contents of all of which are incorporated by this reference), it has now become clear that at the level of infection, the recombinant virus can still sort only a limited effect due to cellular processes that may not be restricted to the serotype used or the presence of neutralizing antibodies raised during earlier infections.

Several studies have indicated that direct application of adenovirus results in a potent immune response against the inserted antigen. In this it does not seem to matter what route of administration is applied. Importantly however, only a few studies have addressed the contribution of diverse cell types in eliciting a B-cell or T-cell-mediated immune response (Shi et al. 2001; Xiang et al. 2002). The intramuscular approach is the most common immunization route used in the art. Cell types available in skeletal muscle are mainly muscle fibers or myocytes, endothelial cells and fibroblasts and to a much lesser extend myoblasts, dendritic cells and macrophages.

In one study, the contribution of the diverse cell types in eliciting immune responses was investigated by culturing and transducing endothelial cells, dendritic cells or skeletal muscle cells ex vivo and subsequently transplanting fixed numbers of transduced cells in syngeneic mice (Mercier et al. 2002). Through follow-up of the mice in terms of immune responses against the inserted antigen (in this particular case β-Gal) it was reported that all cell types were able to induce an antibody response against β-Gal but only the mice transplanted with dendritic cells were able to elicit also CD8⁺-T cell responses against β-Gal. Such studies clearly indicate that dendritic cells play an important role in inducing B-cell and T-cell-mediated immunity.

Vaccine approaches that aim at inducing both mechanisms of the immune system should consist of biologicals able to deliver the antigen to dendritic cells residing in the tissue of vaccine administration. For adenovirus serotype 5 (Ad5, commonly used for gene therapy and vaccination), it is known that high viral loads are required to transduce both primate and rodent dendritic cell in order to obtain some expression of the inserted antigen in these cells (Rea et al. 2001). This low susceptibility of dendritic cells for Ad5 has been attributed to the absence or low expression of the Ad5 receptor: the Coxsackie Adenovirus Receptor (CAR). Adenoviral vectors with a different tropism such that the vector is able to efficiently transduce the dendritic cells are now known in the art: chimeric adenoviral vectors that acquired a novel tropism due to coat-protein swaps (for instance fiber, hexon, penton and/or fiber parts) and adenovirus serotypes that have a natural tropism that is different from the Ad5 tropism, such as Ad11, Ad35 and other serotypes from other subgroups than subgroup C (Farina et al. 2001; Havenga et al. 2002; Mastrangeli et al. 1996; Rea et al. 2001).

It is generally appreciated in the art that dendritic cells are pivotal to evoke a potent immune reaction against foreign antigens (Guermonprez et al. 2002; Lanzavecchia et al. 2001). Dendritic cells are present in different stages in vivo (mature and immature), which stages can be mimicked in vitro by culturing cells in tissue culture plates in the presence of a defined set of growth factors such as IL-4 and GM-CSF. The gathered knowledge accumulated to date concerning immunology dictates that immature dendritic cells are antigen scavengers and thus efficiently capture and process the antigen. Upon capture of an antigen the maturation pathway is induced. During this process, an up-regulation of the number of peptide-MHC complexes on the surface, increased expression of co-stimulatory molecules, and migration of the cells towards the lymphoid organs is observed. Upon full activation by the help of CD4+ T-cells, naïve CD8⁺ T-cells are stimulated by the dendritic cells leading to a Cytotoxic T-Lymphocyte (CTL) population, primed to kill all cells in the body containing the antigen (Lanzavecchia 1998). The CTLs having acquired their “killing status” induce apoptosis in their target cells via at least two mechanisms: one results in DNA fragmentation through uptake of a CTL-produced protein named GranzymeB and the other involves cross-linking of death receptors. Both mechanisms are extremely rapid and are significantly induced during dendritic cell-mediated activation of naïve T-cells (Liu et al. 1989). This latter phenomenon has raised the question whether the dendritic cells themselves are killed once they have activated a CTL. If so, this would be extremely inadequate, potentially resulting in a severely limited T-cell response towards antigens. Importantly, it was actually found in in vitro studies that virus-infected dendritic cells appeared susceptible to CTL killing after several days of infection (Knight et al. 1997).

In contrast to this phenomenon, the general idea in the art is that viruses are entities that use a wide variety of methods to hide from the immune system and to circumvent immune responses towards the infected host cell. Many regions in viral genomes have been identified that encode proteins that play a role in such processes. For instance, adenovirus contains an early expressed region, E3, from which some proteins can for instance down-regulate cellular MHC molecule expression in an attempt to downplay the host immune response. The general view in the art is that viruses, such as adenoviruses, are evolutionary developed to evade the host immune response to a certain level.

An interesting virus-induced-immunosuppression study was performed by Borrow et al. (1995), in which the Choriomeningitis virus (LCMV) was used in mice, being the natural host for the virus. This study demonstrated that inoculation of adult mice with LCMV strain “Armstrong” resulted in rapid clearance of the particles, whereby the mice remained immunocompetent. In contrast, when mice were inoculated with a variant LCMV strain, depicted as “clone 3” (which differs from the parent strain by two amino acids) resulted in persistent infection and obviously in immune suppression. Subsequent biological analyses of the two LCMV strains showed that LCMV clone 3, in contrast to strain Armstrong, was able to infect dendritic cells very efficiently, resulting in clearance of these cells. The increased clearance of dendritic cells infected with LCMV clone 3 resulting in persistent virus infection was clearly correlated with CD8⁺ T-cell-mediated killing. Apparently, LCMV clone 3 differs in its tropism for dendritic cells as compared to the Armstrong strain and is indicative for the key role of tropism in the viral pathogenicity. Clearly, some viruses are able to establish a persistent infection by rapidly killing dendritic cells. In light of the findings described herein it is well possible that LCMV clone 3, like adenovirus serotypes having a tropism for dendritic cells, influence the ability of CD8⁺ cells to efficiently kill the dendritic cells and thus have developed an immune evasion strategy different from non-dendritic cell targeting viruses.

### SUMMARY OF THE INVENTION

The present inventors realized that a viral infection of a dendritic cell may result in an increased Cytotoxic T-Lymphocyte (CTL) sensitivity of that dendritic cell. Thus, when a dendritic cell-specific recombinant adenovirus (for instance Ad35 or a chimeric virus such as Ad5.fib35) is used to infect a dendritic cell, that although this cell was at first relatively resistant towards CTL killing, the infected dendritic cell will most likely be cleared quickly by CTLs due to effects induced by the adenoviral infection. This clearly poses a new potential problem in the quest for an efficient and strong pharmaceutical composition as far as (tumor-) vaccination is concerned. Presumably, one would desire a longer expression of the immunogenic transgene in the dendritic cells to yield a more potent vaccine. Interestingly, on the other hand, for gene therapy purposes, it is clearly advantageous to have a very limited immune response towards the viral vector that is used as a gene delivery vehicle in the pharmaceutical composition. It is actually desired to raise the longevity of expression of the transgene in the target tissue of interest, which is different from the dendritic cell. For this, it would be beneficial to have a swift response of the dendritic-cell-CTL cascade to ensure a low immune response towards the viral vector that is applied. In conclusion, whereas a vaccine may desire a high CTL response and a longer presence of antigen presentation of the infected dendritic cell, it may be beneficial for a gene therapy pharmaceutical to have a quick CTL response against dendritic cells presenting viral antigens, resulting in a lowered immune response towards the proteins of the vehicle and therefore a prolonged presence and activity of the transgene provided by the vehicle, which has infected the tissue of interest. A further application is in targeting unwanted cells using viral vectors carrying therapeutic genes. It is proposed to include in a vector carrying a therapeutic nucleic acid sequence a heterologous nucleic acid sequence, which when expressed, down-regulates PI-9 in the cells in order to sensitize the cells for CTL-related killing.

To overcome at least some of the problems envisaged herein, the present invention provides novel gene delivery vehicles such as viral vectors capable of delivering heterologous nucleic acid to a cell receptive to the gene delivery vehicle, wherein the gene delivery vehicle comprises a (heterologous) nucleic acid which, when expressed in the cell, causes levels of Protease Inhibitor-9 (PI-9) in the cell to be increased or to be maintained at an essentially stable level. The present invention provides also novel gene delivery vehicles such as viral vectors capable of delivering heterologous nucleic acid to a cell receptive to the gene delivery vehicle, wherein the gene delivery vehicle comprises a nucleic acid which, when expressed in the cell, causes the functionality of PI-9 in the cell to be increased or to be maintained at an essentially stable level.

In another embodiment, the invention provides novel gene delivery vehicles such as viral vectors capable of delivering heterologous nucleic acid to a cell receptive to the gene delivery vehicle, wherein the gene delivery vehicle comprises a (heterologous) nucleic acid which, when expressed in the cell, causes levels and/or functionality of PI-9 in the cell to be decreased. The gene delivery vehicles of the present invention are useful in many therapeutic settings such as gene therapy and/or vaccination.

Furthermore, the present invention provides methods of regulating CTL-sensitivity of a cell, comprising the step of: infecting the cell with a gene delivery vector of the present invention.

### BRIEF DESCRIPTION OF THE FIGURES

FIG. 1: CD14⁺ cells (monocytes), immature DC (thick grey line) and mature DC (thin black line), and immature and mature DC infected with adenovirus were stained with 6 different α-human mAbs as indicated. Monocytes only expressed CD14. Differentiation of monocytes into immature DC is evidenced by loss of CD14, and expression of CD86, HLA ABC (MHC class I), and HLA DR (MHC class II). Maturation of DC is evidenced by induction of CD83 expression, and increased expression of CD86, HLA ABC and HLA DR. Adenoviral infection showed no significant differences in expression levels.

FIG. 2: CTL killing of mature (open symbols) and immature (closed symbols) dendritic cells. (A) Mature dendritic cells loaded with peptide were only moderately killed whereas immature dendritic cells were susceptible to CTL-mediated killing. (B) Both mature and immature dendritic cells were highly susceptible to killing when infected with an adenoviral vector.

### DETAILED DESCRIPTION OF THE INVENTION

The present invention provides a solution to at least some of the problems outlined above and to prevent an increased CTL sensitivity of dendritic cells infected with a viral vector during vaccination and to ensure a decreased immune response towards the viral vector used as a gene delivery vehicle in gene therapy applications. For this, the invention provides a viral vector capable of delivering foreign DNA to a cell receptive to the foreign DNA, the viral vector comprising a nucleic acid sequence which, when expressed in the cell, causes levels of PI-9 to be increased or to maintain at a stable level. In another embodiment the present invention provides a viral vector capable of delivering foreign DNA to a cell receptive to the foreign DNA, the viral vector comprising a nucleic acid sequence which, when expressed in the cell, causes levels of PI-9 to be decreased.

The present invention provides a gene delivery vehicle such as a viral vector capable of delivering heterologous nucleic acid to a cell receptive to the viral vector, wherein the viral vector comprises a heterologous nucleic acid sequence which, when expressed in the cell causes levels and/or the activity of Protease Inhibitor-9 (PI-9) in the cell to be increased or to be maintained at an essentially stable level. In a preferred embodiment, the viral vector of the present invention is selected from the group consisting of: adenovirus, alphavirus, poxvirus, vaccinia virus, Human Immunodeficiency Virus (HIV) and influenza virus. In a more preferred embodiment, the viral vector is a (recombinant) adenovirus, or a functional derivative thereof. Even more preferred is an adenovirus serotype from subgroup B, wherein the adenovirus serotype from subgroup B is selected from the group consisting of: Ad11, Ad26, Ad35 and Ad50. Highly preferred are recombinant adenoviral vectors that encounter low levels of neutralizing antibodies due to earlier infections with wild-type or recombinant adenoviruses of that specific serotype. Examples of such adenoviruses are described in PCT International Publication WO 00/70071, wherein functional equivalents of Ad35 are adenovirus serotypes which like Ad35, encounter pre-existing immunity in less than about 10% of the hosts to which it is administered for the first time, or which is capable in more than about 90% of the hosts to which it is administered to avoid or diminish the immune response.

In a specific aspect of the invention, the recombinant adenovirus of the present invention comprises a chimeric capsid comprising proteins and/or protein fragments from at least two different adenovirus serotypes. It is known in the art that for the purpose of targeting recombinant viral vectors such as adenoviruses to specific target cell, one can apply fragments from different adenovirus serotypes, wherein the backbone serotype carries for instance a heterologous nucleic acid (e.g., a therapeutic gene) and the capsid is composed of fragments from two or more adenoviral serotypes. Examples of such “chimeric” vectors are adenoviruses derived from adenovirus serotype 5 (Ad5) carrying a full length or fragment of a fiber from for instance Ad11, Ad16 or Ad35. Such vectors, also known as Ad5.fib11, Ad5.fib16 or Ad5.fib35 can for instance be used to target the adenoviral vector to antigen-presenting cells (APCs) such as dendritic cells. It is now known in the art that numerous swaps can be generated and applied for targeting a wide range of different target tissues and -cells, including tumor cells, APCs, smooth muscle cells, lung cells, certain blood cells and epithelial cells. Thus, in one embodiment, the invention provides a viral vector according to the invention, wherein the cell is an APC. In a preferred embodiment the cell is a dendritic cell or a B-cell, wherein the dendritic cell is immature or mature. Other examples of dendritic cells that can be targeted are peri-arterial inter-digitating dendritic cells.

The present invention also provides viral vectors according to the invention, wherein the heterologous nucleic acid comprises a nucleic acid encoding PI-9, or a functional equivalent thereof. Functional equivalents may be derivatives, fragments, parts, mutants, etc., that are still functional in controlling and/or regulating the CTL-sensitivity of cells in which PI-9 or the equivalent is normally expressed. It is to be understood that if homologues of PI-9 exist, that function in a similar way to regulate CTL-sensitivity, that these functional (and/or sequential homologues) can be used in similar gene delivery vehicles to reach the goals as discussed herein.

In another embodiment, the present invention provides a viral vector capable of delivering heterologous nucleic acid to a cell receptive to the viral vector, wherein the viral vector comprises a heterologous nucleic acid sequence which, when expressed in the cell causes levels and/or functionality of PI-9 in the cell to be decreased. Preferably, such a viral vector comprises a nucleic acid encoding anti-sense PI-9 RNA, or a functional equivalent thereof. Any nucleic acid that decreases the level and/or the functionality of PI-9 in a targeted cell is a functional equivalent. It is to be understood that if genes exist encoding factors that inhibit the function of PI-9 and/or that decrease the protein level of PI-9, that such genes can also be applied in the viral vectors of the present invention to reach the goals as discussed herein.

In yet another embodiment, the invention provides a pharmaceutical composition comprising: a viral vector according to the invention; and a pharmaceutically acceptable carrier. Furthermore, the invention provides a dendritic cell contacted and/or infected with a gene delivery vehicle according to the invention.

In another aspect of the invention, a method of regulating CTL-sensitivity of a cell is provided, the method comprising the step of: infecting the cell with a viral vector comprising a heterologous nucleic acid sequence which, when expressed in the cell, causes the level and/or the activity of PI-9 in the cell to be increased or to be maintained at an essentially stable level, while in another aspect a method of regulating CTL-sensitivity of a cell is provided, the method comprising the step of: infecting the cell with a viral vector comprising a heterologous nucleic acid sequence which, when expressed in the cell, causes the level and/or the activity of PI-9 in the cell to be decreased.

Several methods can be envisioned to increase or decrease the levels and/or functionality of PI-9 and/or equivalents thereof in cell sensitive to CTL killing. A preferred method is to use gene delivery vectors. Examples of gene delivery vectors are viral vectors and micro-particles comprising the nucleic acid to be delivered. Examples of viral vectors are DNA- as well as RNA viruses. Therefore, the nucleic acid that is delivered to the cell of interest according to the present invention may be RNA as well as DNA.

“Heterologous” as used herein means all nucleic acid that is foreign to the gene delivery vector in which it is incorporated. For instance, if a “heterologous” nucleic acid is cloned into an adenoviral genome, this means that the heterologous nucleic acid is not present in the wild-type version of that particular adenovirus. Examples of heterologous nucleic acids are therapeutic genes, such as genes encoding tumor-antigens, immunogenic antigens from pathogenic entities such as bacteria and viruses, apoptosis inducing proteins, hormones and cytokines. Heterologous nucleic acid may also be derived from a different serotype than the serotype in which it is incorporated.

The human Protease Inhibitor-9 (PI-9) and many of its sequence-, distribution-, and functional homologues, such as the mouse Serine Protease Inhibitor-6 (SPI-6) are known in the art (Sprecher et al. 1995; Sun et al. 1997). SPI-6 is a member of the serpin protein family. The protein is most likely able to specifically inactivate GranzymeB (see below) thereby preventing CTL-induced apoptosis. Recent studies have shown that immature dendritic cells derived from mouse and humans are susceptible to CTL killing but, upon maturation, the ability of CTLs to destroy the dendritic cell is lost (Medema et al. 2001). It is hypothesized that a cellular mechanism exists that is able to protect the dendritic cells from CTL-mediated killing. This decrease in CTL-sensitivity has been experimentally attributed to an increase in expression of SPI-6 in the mouse dendritic cells. Although it was elucidated that naïve mature dendritic cells are protected against CTL-cell killing due to up-regulation of SPI-6/PI-9, one interesting question concerns the biological pathway accompanying the up and down regulation of SPI-6/PI-9. As mentioned above, it can be a protein derived from the virus that directly influences the cellular expression of SPI-6/PI-9 or it can be a stress response from the dendritic cell leading to up-, and down regulation of the protease inhibitors.

CTL killing of dendritic cells is brought about by at least two mechanisms: The first mechanism includes the release of the pore-forming molecule, perforin and the cytotoxic protease, GranzymeB (Heusel et al. 1994; Kagi et al. 1994). Upon secretion by the CTL, GranzymeB binds to the mannose-6-phosphate receptor and is taken up by the target cell via receptor-mediated endocytosis (Motyka et al. 2000). Release of GranzymeB from the endosome to the cytoplasm of the target cell occurs via perforin (Shi et al. 1997). Once GranzymeB is present in the cytosol it induces apoptosis through cleavage of cellular substrates such as caspase-3, caspase-7, caspase-8, and Bid (inhibitor of caspase activated DNAase) resulting in DNA fragmentation (Trapani et al. 2000). The second mechanism utilized by a CTL to kill its target is via cross-linking of death receptors. This process involves Fas-Fas Ligand interactions that induce the caspase pathway leading to killing of the target cell (Berke 1995).

As mentioned above, several gene delivery vehicles, such as recombinant adenoviruses are being developed for gene therapy as well as for vaccination purposes. For vaccination, one thrives at boosting the immune response towards inserted antigens to obtain an efficient T-cell reaction against the antigen. For this strategy, an impaired T-cell response to the vector could also result in an impaired, or sub-optimal, T-cell response against the antigen. Next to this direct in vivo vaccination approach, adenoviral vectors can be used to load antigen ex vivo onto dendritic cells derived from patients, whose immune response is inadequate to control tumor cells or infections (Dhodapkar et al. 1999; Steinman et al. 2001; Zhong et al. 2000). After loading the dendritic cells with the antigen of interest, the cells are re-infused in the patient for boosting tumor or virus-specific immunity. A recently recognized problem is that infused antigen loaded dendritic cells are a target for CTL attack (Hermans et al. 2000), and that secondary infusions are less efficient, possibly due to CTL-mediated killing of infused dendritic cells, frustrating the reactivation and boosting of memory T cell responses (Ludewig et al. 2001; Ribas et al. 2000; Ronchese et al. 2001). Clearly, CTL activation by dendritic cells allowed over a period of several days results in more T-cells and in a broader T-cell repertoire against the antigen as compared to an activation period that lasts for only a few hours. The same is most likely true for CTL activation due to repeated (second or more) administrations. Again the experimental findings described here can thus be used to obtain adenoviral-based vaccine delivery vehicles more potent in eliciting a T-cell response against an inserted antigen. For example, one can generate recombinant adenoviral vectors carrying in their genome the gene encoding SPI-6 or the human homologue PI-9, by placing this nucleic acid under the control of a strong eukaryotic promoter. Within several hours of infection of the adenovirus in immature dendritic cells, high levels of PI-9 (or SPI-6) protein are obtained. This increased expression then serves two purposes: (i) it protects mature dendritic cells from CTL-mediated killing by reducing the activity of (for instance) GranzymeB and (ii) it allows survival of immature dendritic cells containing the virus against otherwise efficient killing since immature dendritic cells are not protected. Both mechanisms ensure that more dendritic cells are available to prime naïve T-cells and ensure an increased life span of mature dendritic cells, both mechanisms ultimately resulting in an increased number of T-cells. Another way to ensure that PI-9 levels are not decreased upon viral infection is to determine the mechanism used by the virus to decrease such levels. These mechanisms might be direct or indirect but a proper way to shut-off this mechanism is to apply a recombinant virus lacking the functional regions or domains involved in PI-9 transcription/translational control. Therefore, the present invention discloses at least two ways to ensure sufficiently high levels of PI-9 protein and thereby to protect the infected dendritic cell from clearance through CTL killing: one is to include a PI-9 encoding nucleic acid or a homologue thereof (such as SPI-6) under the control of a strong promoter in the viral backbone, and the second is to delete or impair the functionality of the region in the viral backbone that is involved in down-regulating PI-9 expression of the infected cell. Combinations of the two different mechanisms are also part of the invention.

Within the field of gene therapy, many different vector systems are under investigation for potential treatment of many different lethal human diseases including genetic storage disorders (Gaucher, Pompe, Hurler, Scheie, Hunter, Sanfilippo's, Moquio's, Farber, Niemann-Pick, Krabbe, metachromatic leucodystrophy, thallassemias etc), cardiovascular disorders and applications (stenoses, restenoses, vein grafting), and arthritis, to name but a few. For efficient treatment of many of such diseases, one thrives at long-term expression of the transgene. This would therefore require a mechanism which is different from the vaccination approach, namely for vaccination one would require a stronger and long-lasting immune response towards the antigen. In gene therapy, one does not want a strong reaction towards the gene delivery vehicle that is being applied; for this, one would prefer a weak response and a quick disappearance of dendritic cells that are infected with the gene delivery vehicle. If one uses a gene delivery vehicle that infects dendritic cells (due to a natural tropism) and the target tissue cell of interest, one would desire a response through the dendritic cell-CTL pathway that would be as weak as possible. This effect of a weak CTL response can for instance be achieved by one of the following applications of PI-9. One approach to do so is to insert into the recombinant adenovirus a gene, ribozyme, anti-sense and/or other nucleic acid able to efficiently counteract the expression of for instance SPI-6 or the human homologue PI-9 in dendritic cells. Such a strategy could in principle result in degradation and/or decreased expression of SPI-6 or PI-9 a few hours after infection of the adenovirus in immature dendritic cells, resulting in lower levels of SPI-6 or PI-9 gene product in mature dendritic cells. In turn this is expected to result in direct CTL-mediated killing, finally resulting in a severely hampered T-cell response against the therapeutic vector, such as the recombinant adenoviral vector used to deliver the transgene. A PI-9 inhibiting entity, such as an anti-sense nucleic acid may be placed under control of a dendritic cell-specific promoter, such as the CD83 promoter. Alternatively, over-expression of PI-9 or a homologue thereof, for instance through co-expression of the PI-9 gene next to the therapeutic transgene inserted in the gene delivery vehicle, will result in protection of infected target cells from CTL-mediated killing, and prolonged expression of the therapeutic (immunogenic) gene, which would be highly beneficial in vaccination.

In another setting, generally known in the art as “tumor-vaccination,” it would be required to have a strong reaction towards the antigen encoded by a nucleic acid present in the viral backbone. Therefore, it would be beneficial to have an impaired CTL response, and it would therefore be preferred to over-express the PI-9 protein or a functional homologue thereof, rather than a quick CTL-response. This approach is therefore in the present invention considered to be a vaccination purpose, rather than a gene therapy application. As disclosed herein, it was investigated whether the protection against CTL killing was impaired when the cells were infected with an adenoviral vector. It is shown that mature dendritic cells infected with an adenovirus are not protected against CTL killing, and that both mature and immature peptide-loaded dendritic cells are highly susceptible for CTL-mediated killing compared to uninfected peptide-loaded dendritic cells. These experiments thus show that the adenovirus, once inside the mature dendritic cell alters the normal biology of the cell, making it susceptible towards rapid CTL-mediated killing. This clearance of the antigen-presenting cell can result in an impaired T-cell response against the adenovirus and thus represents a novel method utilized by the adenovirus to evade the host immune system. Alternatively, the immune system may use this mechanism to prevent uncontrolled viral replication in dendritic cells that would otherwise escape from CTL-mediated clearance.

As stated above, herein a new immunological phenomenon rendering virus infected mature dendritic cells susceptible to CTL-mediated killing is disclosed. Moreover, novel adenoviral vectors suitable to down-regulate the host T-cell immune response are disclosed as well as novel adenoviral vectors that are more suitable to boost the host T-cell responses, depending on the application.

Over-expression of SPI-6 in experiments involving mice and PI-9 in humans can be achieved in several ways. One way is to clone this particular nucleic acid under the control of a strong promoter in the E3 region of the adenoviral backbone, while the transgene of interest can be cloned in the E1 region. Examples of strong promoters that can be used are the CMV promoter, the SV40 promoter, the PGK-promoter and the RSV promoter. Examples of transgenes are numerous, but they may include cytokines, therapeutic genes, tumor-antigens, antibodies or parts thereof, or any nucleic acid encoding a protein or therapeutic entity in the purpose of tumor-vaccination, anti-pathogen vaccination and/or gene therapy.

As previously mentioned herein, dendritic cells can also be isolated from patients, targeted ex vivo, cultured and re-introduced in the individual from whom the dendritic cells were derived to elicit antigen-specific immune responses (Dhodapkar et al. 1999; Steinman and Dhodapkar 2001; Zhong et al. 2000). Prolongation of the lifespan of loaded dendritic cells potentially enhances vaccination efficiency. Murine dendritic cell-progenitor cells are isolated from bone marrow, and cultured according to protocols known to persons skilled in the art. The dendritic cell differentiation is determined by staining for markers such as CD 11c and/or CD86, to name a few, followed by FACS analysis. The vectors introduce a transgene, for antigen presentation, and the SPI-6 gene (or PI-9) for dendritic cell survival. This can be achieved either by simultaneous infection with two vectors, one carrying antigen (transgene) and the other carrying SPI-6 (PI-9) in the E1 region (or the E3 region) or vice-versa, or by single infection with a vector carrying antigen (transgene) in the E1 domain and PI-9 (SPI-6) in for instance the E3 region or vice-versa, but other regions can also be applied for each of the transgenes, such as antigens or PI-9 genes. In general, experiments that would include the use of mice would require recombinant vectors encoding SPI-6, while it is to be understood that the invention also encompasses recombinant vectors for the treatment of human disease that would therefore require nucleic acid encoding PI-9. As mentioned above, settings that would require the down-regulation of PI-9 or SPI-6 would of course include settings that would specifically target the expression of the species-specific genes.

In another aspect, the invention provides a method for modulating an immune response to an antigen in a system capable of eliciting a dendritic cell-mediated CD8+ T-cell response to the antigen, the method comprising providing the system with the antigen and with a means for modulating antigen-specific CD8+ T-cell-mediated killing of the dendritic cell.

A system capable of eliciting a dendritic cell-mediated CD8⁺ T-cell response to the antigen can be an in vitro system. Dendritic cells and naïve T-cells can be co-cultured in vitro in the presence of the antigen whereby as a result CD8⁺ T-cells are generated specific for the antigen. This in vitro culture can be supplemented in various ways to improve the efficiency of generation of the CD8⁺ T-cells. Such supplements may comprise (parts of) a lymphnode, a thymus, or other support. In a preferred embodiment the system comprises an individual. In this embodiment the invention is used to modulate an immune response, and in particular the generation of antigen-specific CD8⁺ T-cells in the individual.

The antigen may be provided to the system in various ways. In a preferred embodiment the antigen is provided by a viral vector. Viral vectors are particularly suited for delivery of antigens and/or nucleic acid to target cells. As mentioned herein above, there are many types of viral vectors that can be used in a method of the invention. In a particularly preferred embodiment the viral vector comprises an adenovirus vector.

In one embodiment, the immune response is increased by providing a means for decreasing antigen-specific CD8+ T-cell-mediated killing of the dendritic cell. By decreasing antigen-specific CD8⁺T-cell-mediated killing of the dendritic cell the chance of survival of the dendritic cell and thereby the propensity that the dendritic cell takes part in further education of the immune system, is increased. This has the effect that the pool of CD8⁺T-cells specific for the antigen in the system increases. This aspect of the invention is particularly useful in vaccination applications, wherein a potent antigen-specific immune response is desired.

In another embodiment, the immune response is decreased by providing a means for enhancing antigen-specific CD8+ T-cell-mediated killing of the dendritic cell. By enhancing antigen-specific CD8+ T-cell-mediated killing of the dendritic cell, the chance of survival of the dendritic cell and thereby the propensity that the dendritic cell takes part in further education of the immune system, is decreased. This has the effect the pool of CD8⁺T-cells in the system is decreased. This aspect of the invention is particularly useful in so-called gain of function applications. These gain of function applications are (being) typically though not necessarily developed with viral vectors such as for instance in typical gene therapy applications. Gain of function is often achieved by providing a cell with a gene of interest, wherein the gene of interest provides the cell with additional functionality. A product of the gene of interest and products associated with a viral vector can function as antigens in a system of the invention thereby stimulating an immune response specific for the antigen. In gain of function applications the invention can advantageously used to at least in part prevent an undesired immune response against a product of a gene of interest or of an associated viral vector.

CD8⁺T-cell-mediated killing of dendritic cells can be achieved in several ways. Non-limiting examples are given in the introduction and include at least two mechanisms for inducing DNA-fragmentation in dendritic cells. At least one mechanism comprises uptake of a CTL-produced protein named GranzymeB and at least one other mechanism comprises cross-linking of one or more types of death receptors. In the present invention the activity of at least one pathway for CD8⁺T-cell-mediated killing of dendritic cells is modulated a means of the invention. In a preferred embodiment the activity of the GranzymeB-mediated DNA-fragmentation pathway is modulated. This pathway is particularly amenable for manipulation. In one embodiment the available GranzymeB protein for destruction in dendritic cells is modulated. In a preferred embodiment the means for modulating antigen-specific CD8+ T-cell-mediated killing of the dendritic cell comprises a PI-9 protein or a functional equivalent thereof. Advantages of this preferred embodiment are detailed herein above. In another preferred embodiment the means is provided to the dendritic cell by providing the dendritic cell with a nucleic acid comprising at least part of the PI-9 gene, or a derivative and/or analogue thereof. The nucleic acid may comprise any part of the transcribed region of the PI-9 gene. The part typically comprises at least 20 bases of this region. Preferably, consecutive bases. However, depending on the particular design, interruptions are possible such as for instance to accommodate, or interfere with splicing into mRNA. In this embodiment expression of PI-9 in the cell can for instance be modulated down-ward by providing a PI-9 anti-sense nucleic acid, typically of at least 20 nucleotides, or a homologue of the anti-sense capable of hybridizing to PI-9 (m)RNA under conditions in the cell.

In a preferred embodiment, the means comprises a nucleic acid encoding a PI-9 protein or a protease active part, derivative and/or analogue thereof. In this embodiment activity of the GranzymeB pathway is at least in part decreased.

In a preferred embodiment, the means for modulating antigen-specific CD8+ T-cell-mediated killing of the dendritic cell is provided by a viral vector. In a particularly preferred embodiment both the antigen and the means for modulating antigen-specific CD8+ T-cell-mediated killing of the dendritic cell is provided by the same viral vector. In this way dendritic cells can be provided simultaneously with the antigen and the means thereby improving the overall efficiency, and ease of work of a method of the invention. In this embodiment, the viral vector preferably comprises a tropism allowing efficient transduction of dendritic cells.

In another aspect, the invention provides a composition comprising an antigen and a means for modulating antigen-specific CD8+ T-cell-mediated killing of dendritic cells. Further provided is a kit of parts comprising an antigen and a means for modulating antigen-specific CD8+ T-cell-mediated killing of dendritic cells.

In one embodiment, the composition or kit of parts comprises a viral vector, wherein the vector preferably comprises the antigen and/or a nucleic acid encoding the antigen.

The invention further provides a viral vector comprising a means for specifically modulating antigen-specific CD8+ T-cell-mediated killing of dendritic cells. In one embodiment the invention provides a use of a viral vector of the invention for the preparation of a medicament for modulating an antigen-specific CD8⁺T-cell response in an individual. Preferably, a use the modulation is specific for an antigen provided by the viral vector.

The invention is further explained with the aid of the following illustrative examples.

### EXAMPLES

### Example 1

### Protection of Mature Dendritic Cells from CTL-Mediated Lysis

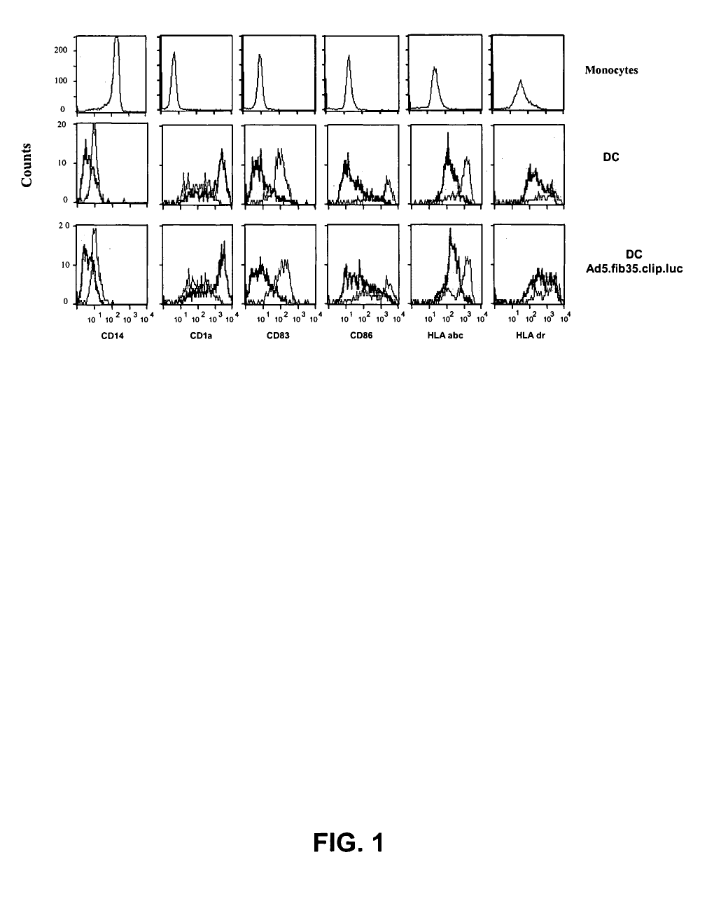

First, experiments were performed to reproduce the findings reported by others on the sensitivity towards CTL-mediated killing of immature dendritic cells and the protection of mature dendritic cells. Hereto, blood from a healthy human individual was isolated and subsequently used to isolate Peripheral blood mononuclear cells through a ficoll step. Using the Variomacs technology known to persons skilled in the art, and CD14-specific antibodies (Miltenyi Biotec GmbH), the monocytes were isolated from the cell population. These monocytes were cultured for 6 days in the presence of 100 ng/ml IL-4 (Bioscource International, Inc.) and 800 IU/ml GM-CSF (Leucomax; Novartis) to obtain immature dendritic cells. Proper differentiation of the monocytes towards immature dendritic cells was monitored via cell surface expression detected with antibodies (from BD Pharmingen) using a FACScalibur apparatus. As known to persons skilled in the art of general immunology, the markers CD11a, CD86, HLA ABC and HLA DR are up-regulated on immature dendritic cells as compared to undifferentiated CD14+ monocytes; mature dendritic cells are distinguished by the expression of CD83. FIG. 1 shows results that were obtained using this isolation/differentiation strategy and moreover gives criteria for quality on which basis it was decided to continue with the cells. Clearly, with the technology used, an essentially pure population of immature dendritic cells was obtained from blood of human individuals.

To artificially mature dendritic cells in vitro, many different stimuli can be added to the culture medium of the immature dendritic cells to obtain such fully matured dendritic cells. These stimuli, that can be used separately, include 200 ng/ml lipopolysaccaride (LPS), 50 μg/ml Poly:IC (both from Sigma), 30% (final volume) monocyte conditioned medium (MCM), CD40L, or 100 ng/ml TNF-α (PreproTech, Inc.). The maturation process was monitored using a FACScalibur and specific antibodies (CD1a, CD14, CD83, CD86, HLA-A, -B, -C and/or -DR). Results are shown in FIG. 1.

After 6 days of culturing, mature and immature dendritic cells were labeled in a mixture of 10 mM Europium, 25 mM DTPA and dextran sulfate (Sigma) for 30 minutes on ice. The cells are recovered by an incubation step of 5 minutes on ice with 100 mM CaCl₂ (Sigma). After this incubation the cells were washed and left for 30-60 minutes at room temperature (RT), and washed once more to clear the largest part of spontaneous Europium leakage.

In the next step cells were loaded with an antigen that is recognized by a CTL clone. Hereto, the well known gp100 melanoma antigen was used in combination with the gp100-specific CTL clone 8J which recognizes gp100 protein both in the context of the mouse K^{b} haplotype as well as the human HLA-A2 haplotype (gift from Dr R. Offringa, Dept. Immunohematology, Leiden University Medical Center, Leiden, NL). Mature or immature dendritic cells were plated in a concentration of 2000 cells/well in 96-well plates using RPMI culture medium. Subsequently, 8J CTLs were added as effector cells to the mature or immature dendritic cells at different effector: target cell ratios: 25:1, 12.5:1 and 6.25:1.

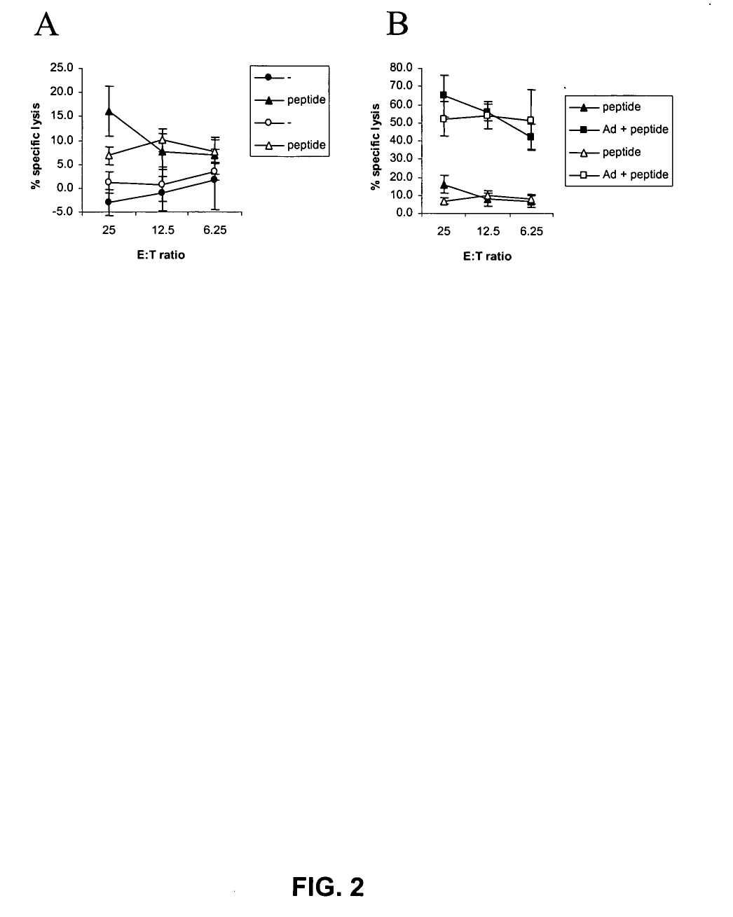

Finally, 10 μg/ml of a synthetically generated peptide (gp100 peptide, amino acids 154-162) was added to the cells, and cells were incubated for 4 hours at 37° C. 20 μl supernatant was harvested and transferred to a flat-bottom microtiter plates (Maxisorp F-96, Nunc) with 200 μl/well Enhancement solution (Wallac/Perkin Elmer Life Science). Release of Europium, indicative for CTL-mediated lysis of mature or immature DC, was detected using a Time Resolved Fluorometer (Wallac/Perkin Elmer Life Science). Results obtained from these experiments are shown in FIG. 2A and demonstrate, as has been described in literature (Medema et al. 2001), that, immature dendritic cells are susceptible for CTL-mediated lysis whereas mature dendritic cells are protected. These results thus validate the assays used in order to investigate viral immune escape mechanisms based on sensitizing antigen presenting cells.

### Example 2

### Adenovirus-Mediated Sensitization of Mature Dendritic Cells

Procedures for obtaining mature and immature dendritic cells from human peripheral blood mononuclear cells, the loading of cells with Europium, and the gp100 peptide loading were generally as described above. The dendritic cells were incubated for 48 hours with replication deficient adenoviral vectors (1000 virus particles per cell (vp/cell)). Controls were not incubated with adenoviral vectors. The adenoviral vector used is based on adenovirus serotype 5 (Ad5), but carries a deletion of the complete adenoviral E1 region. Also, the vector used is engineered to carry the fiber molecule derived from adenovirus serotype 35, giving it a better tropism for dendritic cells. The generation of Ad5 viruses harboring fibers from different (other) serotypes has been extensively described in PCT International Publications WO 00/03029 and WO 02/24730, the contents of both of which are incorporated by this reference. The vector contains within the E1 region a eukaryotic expression cassette consisting of a CMV promoter upstream of a polylinker with at the 3′-end a polyadenylation signal derived from Hepatitis-B virus. The cDNA encoding the firefly luciferase protein, was cloned in the polylinker as described (PCT International Publications WO 00/03029 and WO 00/31285, the contents of both which are incorporated by this reference).

Results of the Europium assay performed on the infected, Europium loaded, and gp100 peptide loaded cells are shown in FIG. 2B. Both mature and immature dendritic cells exposed to adenovirus are extremely sensitive to CTL-mediated killing. These results demonstrate that an adenovirus deleted for E1 (also referred to as a first generation gene transfer vector) sensitizes the dendritic cells resulting in CTL-mediated killing.

To further investigate which adenoviral proteins are involved experiments as described above are repeated using adenoviral vectors that are positive or negative for E1, E2A, E3, E4, or are attenuated in for instance E4 expression. Viruses harboring combinations of the different deletions/mutations, such as E1 and E2A deletions, are also tested. Such viruses can be generated using techniques known to persons skilled in the art and include deletion of such regions from the viral backbone and trans-complementation of the deleted regions in the adenovirus packaging cell lines (PCT International Publications WO 01/05945, WO 01/07571, and WO 01/20014, the contents of which are incorporated by this reference). These experiments allow identification whether early adenovirus proteins are involved in the sensitization of mature dendritic cells.

### Example 3

### Modulation of Mature Dendritic Cell Sensitization by Recombinant Adenovirus

As reported in the literature, up-regulation or level-maintenance of SPI-6 in mice or PI-9 in humans is at least in part involved in the biological pathway used by mature dendritic cells in order to protect against CTL-mediated killing. To investigate whether an adenovirus sensitizes dendritic cells via the PI-9 pathway, two different experimental strategies are performed: (i) Western-blot analyses and/or reverse transcription PCR to determine the protein and/or RNA expression levels of PI-9 in human dendritic cells either exposed to, or not exposed to adenoviral vectors. (ii) Generation of adenoviral vectors carrying the PI-9 cDNA, subsequent infection of mature and immature dendritic cells, followed by determination of the effect of PI-9 over-expression on CTL-mediated killing.

To determine the expression levels of PI-9, human monocyte derived mature and immature dendritic cells are obtained as described above. The cells are seeded at a concentration of 2.5×10⁶ cells per well in three 6-well plates. Next, no virus or 1000 vp/cell of Ad5.Luciferase (negative control) or Ad5.PI-9 virus is added and infection is allowed to proceed for 24 hours (both mature and immature human dendritic cells are used and each parameter in triplicate). After 24 h, cells of each well are harvested and used for the Europium cell lysis assay (one well), to generate a protein lysate (one well), and to isolate RNA from the cell population (one well) for reverse transcriptase PCR. The Europium cell lysis assay is performed as described above. A western blot analysis to determine the PI-9 protein expression level is performed by loading 10 μg total protein on protein separation gels (Invitrogen, NPO321, Nupage 4-12% Bis-Tris Gel or Biorad, Criterion gels), using the instructions provided by the manufacturers. Proteins are transferred to Immunobilon-P PVDF membranes (pore size: 0.45 μm). The membranes are blocked for 1 hour in blocking buffer (5% milk powder, and 0.1% Tween-20 in PBS). To visualize the PI-9 protein, the antisera MoAb17 and/or PI-9-K are used: Mouse monoclonal antibody MoAb17 (subtype IgG1) is specific for the human PI-9 protein and efficiently cross-reacts with mouse SPI-6 protein (Bladergroen et al. 2001). For detection of PI-9 protein, MoAb17 are diluted in PBS (3.6 μg/ml), added to the western blot, and incubated for 3 hours at RT. Then, the antibody solution is removed by washing the blots twice with 10 ml of PBS. Binding of MoAb17 to the blot is visualized using horse-radish-peroxidase (HRP)-labeled secondary anti-mouse IgG1 antibody and the ECL detection system according to the instructions provided by the manufacturer (Amersham) and using general methods know to the skilled person.

To determine PI-9 expression levels with reverse transcription PCR, total RNA is isolated from 10⁶ cells per population using TRIzol (Life Technologies) following the instructions provided by the manufacturer. Obtained RNA is dissolved in RNAse free water and incubated with DNAse (Life technologies) according to the manufacturer's directions. Then, the RNAse solution is incubated at 70° C. for 10 minutes in the presence of 1 μg oligo dT primers and 100 ng random hexamer-primers, in a total volume of 15 μl. RNA is kept on ice and dNTP, DTT, 5 μl first strand buffer, and 1 unit RNAse H-superscript II Reverse transcriptase was added (all reagents obtained from Invitrogen), and incubated at 42° C. for 50 minutes. The reaction is stopped by incubation at 70° C. for 10 minutes.

For PI-9-specific PCR, 1 μl of cDNA solution is used in a PCR reaction. Forward (PI-9F: 5′-GGC ATT TGG GAA TTG TTG ATG-3′ (SEQ ID NO:______)), and reverse (PI-9R: 5′-TGG CGA TGA GAA CCT GCC AC-3′ (SEQ ID NO:______)) primers, located within one exon, are added in a concentration of 10 pmol per primer per reaction. Furthermore, per reaction is added: 2 mmol dNTP, 1x MgCl₂, 2.5 U Taq polymerase, 1x Taq buffer (all from Invitrogen), and H₂O to a volume of 50 μl. PCR reactions are generally carried out using the following basal program: 5 minutes at 94° C. followed by 35 cycles, each consisting of 1 minute at 94° C., 30 seconds at 60° C., and 1 minute at 72° C. After 20 cycles, every 3 cycles, 5 μl of the PCR product is aliquoted from the reaction tube and analyzed by agarose gel electrophoresis to determine semi quantitatively the amount of PI-9 PCR product.

To determine whether over-expression of the PI-9 protein in mature and immature dendritic cells results in protection against CTL killing of otherwise sensitive cells, it is useful to infect such immature and mature dendritic cells with an adenovirus harboring the human PI-9 cDNA. First, the PI-9 cDNA has to be cloned. For this, total RNA is isolated from approximately 5×10⁵ matured human monocyte derived dendritic cells using the TRIzol reagent (Life Technologies) and the instructions provided by the manufacturer. cDNAs are generated from this cellular RNA via reverse transcription PCR using the random hexamer RT-PCR kit (Applied Biosystems, N808-0234) following the instructions provided by the manufacturer. Next, the cDNA is used as a template to amplify the complete coding domain of human PI-9 by PCR. Hereto, oligonucleotides PI-9Forw (5′-GGG GTA CCG CCA CCA TGG AAA CTC TTT CTA AGT GG-3′ (SEQ ID NO:______)) and PI-9Rev (5′-CGG GAT CCT TAT GGC GAT GAG AAC CTG C-3′ (SEQ ID NO:______)) are synthesized (InvitroGen). PI-9Forw contains a KpnI restriction site and PI-9Rev contains a BamHI restriction site. The following PCR program is applied: 5 minutes at 94° C., followed by 30 cycles, each consisting of 1 minute at 94° C., 30 seconds at 59° C., and 1 minute at 72° C. The PCR reaction is terminated by 10 minutes incubation at 72° C. 5 μl of the PCR product is analyzed by agarose gel electrophoresis. The PCR amplification product is a fragment of 1153 base pairs. Subsequently, 20 μl PCR product is digested with restriction enzymes KpnI and BamHI, as is plasmid pAdapt (PCT International Publication WO 00/63403, the contents of which are incorporated by this reference). Briefly, pAdapt contains approximately 5000 base pairs of the left end of the Ad5 genome. Within this adenoviral region, the domain encoding for E1 proteins is removed entirely and replaced by a eukaryotic expression cassette comprising the CMV promoter and a BGH poly(A). The KpnI/BamHI digested PI-9 nucleic acid is subsequently cloned into the KpnI/BamHI digested pAdapt plasmid using general methods known to the person skilled in the art of molecular biology. Correct insertion into pAdapt and correct amplification of the human PI-9 coding sequence is confirmed by restriction digests patterns and/or sequencing. To obtain a recombinant adenovirus carrying a PI-9 nucleic acid (wherein the virus has a tropism for human dendritic cells), the pAdapt plasmid carrying the PI-9 sequence is first digested with PacI to free the viral sequence from the plasmid and subsequently transfected into packaging cells such as PER.C6™ cells together with a PacI digested cosmid containing the remaining 31,000 base pairs of the right end of the Ad5 genome (see, for details of these procedures, PCT International Publication WO 99/55132, the contents of which are incorporated by this reference). The tropism for dendritic cells is provided by either replacing a fiber part of the Ad5 backbone for a fiber part from another serotype harboring the dendritic cell-specific tropism, or by using an adenovirus serotype that has a natural affiliation for cells such as dendritic cells. Examples of adenoviruses that have a tropism for, for instance, dendritic cells are the adenovirus serotype 11 and 35 (Ad11 and Ad35). This example is limited to the use of Ad5fib35.PI-9 chimeric viruses, but other experiments are performed in which Ad11 and/or Ad35 backbones are used, wherein these adenoviral vectors harbor the nucleic acid encoding the PI-9 protein. In these cases it is not necessary to swap fibers or fiber parts, or other parts of the viral coat. The use of Ad11, Ad35 and other non-group C adenoviruses for such targeting purposes has been described (PCT International Publications WO 00/70071 and WO 02/40665, the contents of which are incorporated by this reference). After transfection, a functional adenovirus genome is formed, that replicates due to helper functions provided by the packaging cell and that is packaged to form progeny virus, which is released into the culture medium due to lysis of the host cell. Virus production, purification, and titration are performed using technology and protocols well known to persons skilled in the art of adenoviral production. The chimeric virus is coded Ad5.Fib35.PI-9.

Mature and immature dendritic cells are generated as described above. Both mature and immature DC are seeded at a concentration of 1×10⁶ cells per well of 6-well plates and exposed to 1000 vp/cell of either Ad5.Fib35 (lacking a transgene) or Ad5.Fib35.PI-9, using non-infected cells as controls. After 48 hours, cells are harvested and a cell lysate is prepared as described above. Using MoAb17 and western blot analysis the PI-9 protein levels are determined in the mature and immature dendritic cells. To further determine whether up- and down regulation of PI-9 protein expression correlates and thus whether mature and immature dendritic cells are protected against CTL-mediated killing, the experiment described above is performed in parallel, but cells are taken for the Europium cell toxicity assay. Both mature and immature dendritic cells are mainly killed by CTL response when infected with Ad.luc viruses, but survive to a significant extent when viruses are used that carry the PI-9 gene.

### Example 4

### In Vivo Determination of the Potency of the Anti-Adenovirus Humoral and Cellular Responses with Virus Encoding SPI-6

To investigate the in vivo effect of over-expressing PI-9 in immature and mature antigen presenting cells on the anti-adenovirus humoral and cellular responses, also adenoviral vectors are generated carrying the cDNA encoding the mouse homologue of the human PI-9, namely Serine Protease Inhibitor-6 (SPI-6). Hereto, first the SPI-6 cDNA is generated. To PCR amplify the SPI-6 cDNA, total cellular RNA is isolated from mouse dendritic cells or tissues that have high expression of SPI-6 RNA, such as lung, spleen, kidney or heart and subsequently converted to cDNA as described above. The complete coding sequence is amplified by PCR with the primers SPI-6 F (5′-GGG GTA CCG CCA CCA TGA ATA CTC TGT CTG AAG G-3′ (SEQ ID NO:______) and SPI-6 R (5′-CGG GAT CCT TAT GGA GAT GAG AAC CTG CC-3′ (SEQ ID NO:______), generating a 1147 base pair PCR product. This product is cloned into pAdApt after digestion with KpnI and BamHI as described above. Subsequently, the chimeric vector which is named Ad5.Fib35.SPI-6, is used to generate the recombinant non-replicating virus in host cells, such as PER.C6™. Then, viruses are purified and titrated as described above using methods known to persons skilled in the art.

The control virus Ad5.Fib35 and the Ad5.Fib35.SPI-6 virus are injected intramuscularly in separate mice at a dose of 10¹⁰ viral particles. Spleens from these mice are analyzed for T-cell responses and serum for antibody responses against the vector two weeks after injection. These analyses like neutralization assay, IFN-γ production, and intracellular staining, are performed using technology and protocols well known to persons skilled in the art of immunological responses. These experiments allow identification of the role of SPI-6 on evoking humoral and cellular immune responses against adenoviral vectors in vivo.

### Example 5

### Role of SPI-6 in Immunity Against the Viral Vector

Ad5.Fib35 and Ad5.Fib35.SPI-6 are injected intramuscularly in separate mice at a dose of 10¹⁰ viral particles. Two weeks later, 10¹⁰ viral particles of Ad5.Fib35.luc is injected intramuscularly in each mouse, followed by sacrificing groups of mice at day 1, 2, 4, 8, and 12 days and muscle tissues are isolated. Tissues are homogenized in 600 μl cold PO₄ buffer pH 7.8, and subsequently added to 400 μl lysis buffer (PO₄ buffer pH 7.8, 1 mM DTT+0.1% Triton X-100). Lysed muscle samples are freeze-thawed and 20 μl is transferred into white cliniplates (Thermo Lifescience). Plates are placed in a luminoscan (Thermo Lifescience), 20 μl of luciferase substrate (Luciferase-assay systems, Promega) is added and luciferase activity is measured for 10 seconds. The amount of luciferase activity represents the number of muscle cells expressing the transgene. This activity might be decreased due to enhanced immunity against the vector in the Ad5.Fib35.SPI-6 primed mice and thus also compared to prolonged expression in Ad5.Fib35 (no transgene) primed mice. Mice that are pre-injected with the empty vector generate a substantial anti-vector T-cell response. Luciferase activity in injected muscle increases shortly and after a few days decreases due to T-cell elimination of adenovirus-infected muscle cells. In contrast, mice pre-injected with Ad5.fib35.SPI-6 are predicted to mount a weaker vector-specific T-cell immunity as compared to the control mice, and are predicted to have a prolonged expression of luciferase in muscle cells.

In conclusion, using viral vectors carrying a heterologous nucleic acid encoding a target antigen in combination with a nucleic acid encoding PI-9 under the control of a strong promoter, such as a CMV promoter, in targeting a antigen presenting cell, would result in overexpression of PI-9 during the expression of the target antigen in that cell which would normally be sensitized towards CTL killing. This overexpression would then prevent the cell being removed by CTL-response and therefore result in a longer (and most likely stronger) immune response towards the target antigen. This would be highly beneficial in vaccination applications.

In contrast, using viral vectors in gene therapy applications (for instance for targeting tumor cells), wherein these viral vectors carry a gene of interest encoding a therapeutic protein in combination with a nucleic acid or an entity that down-regulates PI-9 expression, might result in a decreased immune response towards the viral vector that is being applied, because antigen presenting cells presenting antigens from the virus, would be eliminated rapidly. This rapid clearance of these viral-antigen presenting cells would then result in a stronger and prolonged effect towards the tumor cell being targeted.

### REFERENCES

Berke G. (1995). The CTL's kiss of death. Cell 81:9-12.
Bladergroen B. A., Strik M. C., Bovenschen N., van Berkum O., Scheffer G. L., Meijer C. J., Hack C. E. and Kummer J. A. (2001). The granzyme B inhibitor, protease inhibitor 9, is mainly expressed by dendritic cells and at immune-privileged sites. J. Immunol. 166:3218-25.
Borrow P., Evans C. F. and Oldstone M. B. (1995). Virus-induced immunosuppression: immune system-mediated destruction of virus-infected dendritic cells results in generalized immune suppression. J. Virol. 69:1059-70.
Bruna-Romero O., Gonzalez-Aseguinolaza G., Hafalla J. C., Tsuji M. and Nussenzweig R. S. (2001). Complete, long-lasting protection against malaria of mice primed and boosted with two distinct viral vectors expressing the same plasmodial antigen. Proc. Natl. Acad. Sci. U.S.A. 98:11491-6.
Dhodapkar M. V., Steinman R. M., Sapp M., Desai H., Fossella C., Krasovsky J., Donahoe S. M., Dunbar P. R., Cerundolo V., Nixon D. F. and Bhardwaj N. (1999). Rapid generation of broad T-cell immunity in humans after a single injection of mature dendritic cells. J. Clin. Invest. 104:173-80.
Farina S. F., Gao G. P., Xiang Z. Q., Rux J. J., Burnett R. M., Alvira M. R., Marsh J., Ertl H. C. and Wilson J. M. (2001). Replication-defective vector based on a chimpanzee adenovirus. J. Virol. 75:11603-13
Guermonprez P., Valladeau J., Zitvogel L., Thery C. and Amigorena S. (2002). Antigen presentation and T cell stimulation by dendritic cells. Annu. Rev. Immunol. 20:621-67.
Havenga M. J., Lemckert A. A., Ophorst O. J., van Meijer M., Germeraad W. T., Grimbergen J., van Den Doel M. A., Vogels R., van Deutekom J., Janson A. A., de Bruijn J. D., Uytdehaag F., Quax P. H., Logtenberg T., Mehtali M. and Bout A. (2002). Exploiting the natural diversity in adenovirus tropism for therapy and prevention of disease. J. Virol. 76:4612-20.
Hermans I. F., Ritchie D. S., Yang J., Roberts J. M. and Ronchese F. (2000). CD8+ T cell-dependent elimination of dendritic cells in vivo limits the induction of antitumor immunity. J. Immunol. 164:3095-101.
Heusel J. W., Wesselschmidt R. L., Shresta S., Russell J. H. and Ley T. J. (1994). Cytotoxic lymphocytes require granzyme B for the rapid induction of DNA fragmentation and apoptosis in allogeneic target cells. Cell 76:977-87.
Kagi D., Vignaux F., Ledermann B., Burki K., Depraetere V., Nagata S., Hengartner H. and Golstein P. (1994). Fas and perforin pathways as major mechanisms of T cell-mediated cytotoxicity. Science 265:528-30.
Knight S. C., Askonas B. A. and Macatonia S. E. (1997). Dendritic cells as targets for cytotoxic T lymphocytes. Adv. Exp. Med. Biol. 417:389-94.
Lanzavecchia A. (1998). Immunology. Licence to kill. Nature 393:413-4.
Lanzavecchia A. and Sallusto F. (2001). Regulation of T cell immunity by dendritic cells. Cell 106:263-6.
Liu C. C., Rafii S., Granelli-Pipemo A., Trapani J. A. and Young J. D. (1989). Perforin and serine esterase gene expression in stimulated human T cells. Kinetics, mitogen requirements, and effects of cyclosporin A. J. Exp. Med. 170:2105-18.
Ludewig B., Bonilla W. V., Dumrese T., Odermatt B., Zinkemagel R. M. and Hengartner H. (2001). Perforin-independent regulation of dendritic cell homeostasis by CD8(+) T cells in vivo: implications for adaptive immunotherapy. Eur. J. Immunol. 31:1772-9.
Mastrangeli A., Harvey B. G., Yao J., Wolff G., Kovesdi I., Crystal R. G. and Falck-Pedersen E. (1996). “Sero-switch” adenovirus-mediated in vivo gene transfer: circumvention of anti-adenovirus humoral immune defenses against repeat adenovirus vector administration by changing the adenovirus serotype. Hum. Gene Ther. 7:79-87.
Medema J. P., Schuurhuis D. H., Rea D., van Tongeren J., de Jong J., Bres S. A., Laban S., Toes R. E., Toebes M., Schumacher T. N., Bladergroen B. A., Ossendorp F., Kummer J. A., Melief C. J. and Offringa R. (2001). Expression of the serpin serine protease inhibitor 6 protects dendritic cells from cytotoxic T lymphocyte-induced apoptosis: differential modulation by T helper type 1 and type 2 cells. J. Exp. Med. 194:657-67.
Mercier S., Gahery-Segard H., Monteil M., Lengagne R., Guillet J. G., Eloit M. and Denesvre C. (2002). Distinct roles of adenovirus vector-transduced dendritic cells, myoblasts, and endothelial cells in mediating an immune response against a transgene product. J. Virol. 76:2899-911.
Motyka B., Korbutt G., Pinkoski M. J., Heibein J. A., Caputo A., Hobman M., Barry M., Shostak I., Sawchuk T., Holmes C. F., Gauldie J. and Bleackley R. C. (2000). Mannose 6-phosphate/insulin-like growth factor II receptor is a death receptor for granzyme B during cytotoxic T cell-induced apoptosis. Cell 103:491-500.
Rea D., Havenga M. J., van Den Assem M., Sutmuller R. P., Lemckert A., Hoeben R. C., Bout A., Melief C. J. and Offringa R. (2001). Highly efficient transduction of human monocyte-derived dendritic cells with subgroup B fiber-modified adenovirus vectors enhances transgene-encoded antigen presentation to cytotoxic T cells. J. Immunol. 166:5236-44.
Ribas A., Butterfield L. H., Hu B., Dissette V. B., Meng W. S., Koh A., Andrews K. J., Lee M., Amar S. N., Glaspy J. A., McBride W. H. and Economou J. S. (2000). Immune deviation and Fas-mediated deletion limit antitumor activity after multiple dendritic cell vaccinations in mice. Cancer Res. 60:2218-24.
Ronchese F. and Hermans I. F. (2001). Killing of dendritic cells: a life cut short or a purposeful death? J. Exp. Med. 194:F23-6.
Shi L., Mai S., Israels S., Browne K., Trapani J. A. and Greenberg A. H. (1997). Granzyme B (GraB) autonomously crosses the cell membrane and perforin initiates apoptosis and GraB nuclear localization. J. Exp. Med. 185:855-66.
Shi Z., Zeng M., Yang G., Siegel F., Cain L. J., van Kampen K. R., Elmets C. A. and Tang D. C. (2001). Protection against tetanus by needle-free inoculation of adenovirus-vectored nasal and epicutaneous vaccines. J. Virol. 75:11474-82.
Shiver J. W., Fu T. M., Chen L., Casimiro D. R., Davies M. E., Evans R. K., Zhang Z. Q., Simon A. J., Trigona W. L., Dubey S. A., Huang L., Harris V. A., Long R. S., Liang X., Handt L., Schleif W. A., Zhu L., Freed D. C., Persaud N. V., Guan L., Punt K. S., Tang A., Chen M., Wilson K. A., Collins K. B., Heidecker G. J., Fernandez V. R., Perry H. C., Joyce J. G., Grimm K. M., Cook J. C., Keller P. M., Kresock D. S., Mach H., Troutman R. D., Isopi L. A., Williams D. M., Xu Z., Bohannon K. E., Volkin D. B., Montefiori D. C., Miura A., Krivulka G. R., Lifton M. A., Kuroda M. J., Schmitz J. E., Letvin N. L., Caulfield M. J., Bett A. J., Youil R., Kaslow D. C. and Emini E. A. (2002). Replication-incompetent adenoviral vaccine vector elicits effective anti-immunodeficiency-virus immunity. Nature 415:331-5.
Sprecher C. A., Morgenstern K. A., Mathewes S., Dahlen J. R., Schrader S. K., Foster D. C. and Kisiel W. (1995). Molecular cloning, expression, and partial characterization of two novel members of the ovalbumin family of serine proteinase inhibitors. J. Biol. Chem. 270:29854-61.
Steinman R. M. and Dhodapkar M. (2001). Active immunization against cancer with dendritic cells: the near future. Int. J. Cancer 94:459-73.
Sullivan N. J., Sanchez A., Rollin P. E., Yang Z. Y. and Nabel G. J. (2000). Development of a preventive vaccine for Ebola virus infection in primates. Nature 408:605-9. Sun J., Ooms L., Bird C. H., Sutton V. R., Trapani J. A. and Bird P. I. (1997). A new family of 10 murine ovalbumin serpins includes two homologs of proteinase inhibitor 8 and two homologs of the granzyme B inhibitor (proteinase inhibitor 9). J. Biol. Chem. 272:15434-41.
Trapani J. A., Davis J., Sutton V. R. and Smyth M. J. (2000). Proapoptotic functions of cytotoxic lymphocyte granule constituents in vitro and in vivo. Curr. Opin. Immunol. 12:323-9.
Xiang Z., Gao G., Reyes-Sandoval A., Cohen C. J., Li Y., Bergelson J. M., Wilson J. M. and Ertl H. C. (2002). Novel, chimpanzee serotype 68-based adenoviral vaccine carrier for induction of antibodies to a transgene product. J. Virol. 76:2667-75.
Zhong L., Granelli-Piperno A., Pope M., Ignatius R., Lewis M. G., Frankel S. S. and Steinman R. M. (2000). Presentation of SIVgag to monkey T cells using dendritic cells transfected with a recombinant adenovirus. Eur. J. Immunol. 30:3281-90.

1. A viral vector capable of delivering heterologous nucleic acid to a cell receptive to said viral vector, said viral vector comprising:
a heterologous nucleic acid sequence which, when expressed in said cell, modulates levels and/or activity of Protease Inhibitor-9 (PI-9) in said cell so as to increase, decrease, maintain, or decrease the activity or functionality of PI-9.

2. The viral vector of claim 1, wherein said viral vector is selected from the group consisting of adenovirus, alphavirus, poxvirus, vaccinia virus, Human Immunodeficiency Virus, and influenza virus.

3. The viral vector of claim 2, wherein said viral vector is of adenovirus origin.

4. The viral vector of claim 3, wherein said adenovirus is subgroup B adenovirus.

5. The viral vector of claim 4, wherein said adenovirus is selected from the group consisting of Ad11, Ad26, Ad35, and Ad50.

6. The viral vector of claim 3, wherein said adenovirus comprises a chimeric capsid comprising peptides from at least two different adenovirus serotypes.

7. The viral vector of claim 1, wherein said cell is an antigen-presenting cell.

8. The viral vector of claim 1, wherein said cell is a dendritic cell or a B-cell.

9. The viral vector of claim 8, wherein said cell is an immature dendritic cell.

10. The viral vector of claim 8, wherein said cell is a mature dendritic cell.

11. The viral vector of claim 8, wherein said dendritic cell is a peri-arterial inter-digitating dendritic cell.

12. The viral vector of claim 1, wherein said heterologous nucleic acid sequence comprises a nucleic acid sequence encoding PI-9 so as to cause the level and/or the activity of PI-9 in the cell to be increased or to be maintained at an essentially stable level.

13. The viral vector of claim 1, wherein said nucleic acid comprises a nucleic acid encoding anti-sense PI-9 RNA so as to cause the level and/or the activity of PI-9 in the cell to be decreased.

14. A pharmaceutical composition comprising:
the viral vector of claim 1; and
a pharmaceutically acceptable carrier.

15. A dendritic cell comprising the viral vector of claim 1.

16. A method of regulating CTL-sensitivity of a cell, said method comprising:
infecting said cell with a viral vector comprising a heterologous nucleic acid sequence that, when expressed in said cell, regulates the cell's level and/or the activity of PI-9 so as to be increased, decreased, or maintained at an essentially stable level.

17. The method according to claim 16, wherein said viral vector is selected from the group consisting of: adenovirus, alphavirus, poxvirus, vaccinia virus, Human Immunodeficiency Virus, and influenza virus.

18. The method according to claim 17, wherein said viral vector is of adenovirus origin.

19. The method according to claim 18, wherein said adenovirus is an adenovirus serotype from subgroup B.

20. The method according to claim 19, wherein said adenovirus is selected from the group consisting of Ad11, Ad26, Ad35, and Ad50.

21. The method according to claim 18, wherein said adenovirus comprises a chimeric capsid comprising peptides from at least two different adenovirus serotypes.

22. The method according to claim 16, wherein said cell is an antigen-presenting cell.

23. The method according to claim 16, wherein said cell is a dendritic cell or a B-cell.

24. The method according to claim 23, wherein said cell is an immature dendritic cell.

25. The method according to claim 33, wherein said cell is a mature dendritic cell.

26. The method according to claim 23, wherein said dendritic cell is a peri-arterial inter-digitating dendritic cell.

27. The method according to claim 16, wherein said nucleic acid comprises a nucleic acid encoding PI-9 and the level and/or the activity of PI-9 in the cell is increased or maintained at an essentially stable level.

28. The method according to claim 16, wherein said nucleic acid comprises a nucleic acid encoding anti-sense PI-9 RNA and the level and/or the activity of PI-9 in the cell is decreased.

29. A method of modulating an immune response to an antigen in a system capable of eliciting a dendritic cell-mediated CD8+ T-cell response to said antigen, said method comprising:
providing the system with the antigen and further with means for modulating antigen-specific CD8+ T-cell-mediated killing of a dendritic cell.

30. The method according to claim 29, wherein said antigen is provided by a viral vector.

31. The method according to claim 29, wherein said viral vector comprises an adenovirus vector.

32. The method according to claim 29, wherein said immune response is enhanced by providing a means for decreasing antigen-specific CD8+ T-cell-mediated killing of a dendritic cell.

33. The method according to claim 32, wherein said means modulates the GranzymeB-mediated DNA-fragmentation pathway.

34. The method according to claim 33, wherein said means comprises a PI-9 protein.

35. The method according to claim 33, wherein said means is provided to said dendritic cell by providing said dendritic cell with a nucleic acid comprising at least part of the PI-9 gene.

36. The method according to claim 33, wherein said means comprises a nucleic acid sequence encoding a PI-9 protein or a protease active part, derivative and/or analogue thereof.

37. The method according to claim 29, wherein said means for modulating antigen-specific CD8+ T-cell-mediated killing of said dendritic cell is provided by a viral vector.

38. A composition comprising:
an antigen, and
means for modulating antigen-specific CD8+ T-cell-mediated killing of dendritic cells.

39. The composition of claim 38 further comprising:
a viral vector.

40. A viral vector composition comprising:
means for specifically modulating antigen-specific CD8+ T-cell-mediated killing of dendritic cells, and
a pharmaceutically acceptable excipient.

41. The method according to claim 29, wherein said immune response is decreased by providing a means for enhancing antigen-specific CD8+ T-cell-mediated killing of a dendritic cell.

42. The method according to claim 41, wherein said means modulates the GranzymeB-mediated DNA-fragmentation pathway.

43. The method according to claim 42, wherein said means comprises a PI-9 protein.

44. The method according to claim 42, wherein said means is provided to said dendritic cell by providing said dendritic cell with a nucleic acid comprising at least part of the PI-9 gene.

45. The method according to claim 43, wherein said means comprises a nucleic acid sequence encoding a PI-9 protein or a protease active part, derivative and/or analogue thereof.

46. The method according to claim 41, wherein said means for modulating antigen-specific CD8+ T-cell-mediated killing of said dendritic cell is provided by a viral vector.

47. A kit of parts comprising:
an antigen, and, also contained therein, for use by a user,
means for modulating antigen-specific CD8+ T-cell-mediated killing of dendritic cells.
